(19) United States
(12) Patent Application Publication
(10) Pub. No.: US 2026/0224513 A1
(43) Pub. Date: Aug. 6, 2026

(54) TREATMENT OF AGE-RELATED MACULAR DEGENERATION, GLAUCOMA, DIABETIC RETINOPATHY AND GEOGRAPHIC ATROPHY WITH N-ACETYLCYSTEINE AMIDE (NACA) OR (2R,2R')-3,3'-DISULFANEDIYL BIS(2-ACETAMIDOPROPANAMIDE) (DINACA)

(71) Applicant: Nacuity Pharmaceuticals, Inc., Fort Worth, TX (US)

(72) Inventor: G. Michael Wall, Fort Worth, TX (US)

(21) Appl. No.: 19/467,161

(22) Filed: Feb. 2, 2026

Related U.S. Application Data
(63) Continuation-in-part of application No. 18/447,720, filed on Aug. 10, 2023.
Application No. 18/447,720 is a continuation-in-part of application No. 16/738,819, filed on Jan. 9, 2020, now Pat. No. 11,766,413.
(60) Provisional application No. 62/791,396, filed on Jan. 11, 2019.
(60) Provisional application No. 63/753,175, filed on Feb. 3, 2025.

(51) Int. Cl. A61K 31/16; A61K 9/00; A61P 27/02

(52) U.S. Cl. CPC A61K 31/16; A61K 9/0053; A61P 27/02

(57) ABSTRACT

Cataracts are the most common cause of treatable blindness worldwide, and result from loss of transparency of the lens. It has been demonstrated that oxidative stress plays an important role in cataract etiopathogenesis. Oxidative damage reduces solubility of crystallins, the main structural proteins of the lens, resulting in opacification. Cells have evolved to combat this damage with antioxidant enzymes and low molecular weight antioxidants such as glutathione (GSH). Our primary objective was to evaluate a GSH prodrug for cataract treatment because current surgical options are neither economical nor safe. Recent literature indicates that thiol compounds like N-acetylcysteine (NAC) may ameliorate the risk for cataracts. Our data indicates that the analog of NAC, N-acetylcysteine amide (NACA), is more effective than NAC due to its higher bioavailability. Therefore, the present study was performed to determine whether NACA is also effective in sodium selenite and L-buthionine-(S,R)-sulfoxamine (BSO)-induced cataracts. In both models, NACA significantly decreased lens opacity and improved redox balance. The data suggest that NACA has the potential to significantly improve both patient health and the clinical treatment of cataracts.

### CROSS-REFERENCE TO RELATED APPLICATIONS

This application claims priority to U.S. Provisional Application Ser. No. 63/753,175, filed Feb. 3, 2025, entitled “Treatment of Age-Related Macular Degeneration, Glaucoma, and Diabetic Retinopathy with Usher Syndrome Associated with Retinitis Pigmentosa With N-Acetylcystieine Amide (NACA) OR) 2R,2R′)-3,3′-Disulfanediyl Bis (2-Acetamidopropanamide) (DINACA)”, and is a continuation-in-part application of U.S. application Ser. No. 18/447,720 filed Aug. 10, 2023, which is a continuation-in-part of U.S. application Ser. No. 16/738,819 filed Jan. 9, 2020, now issued U.S. Pat. No. 11,766,413, which claims priority to U.S. Provisional Application Ser. No. 62/791,396, filed Jan. 11, 2019, the entire contents of each of which are incorporated herein by reference.

### STATEMENT OF FEDERALLY FUNDED RESEARCH

Not applicable.

### TECHNICAL FIELD

The present disclosure relates in general to the field of compositions and methods for treating age-related macular degeneration, glaucoma, and/or diabetic retinopathy using N-acetylcysteine amide (NACA) or (2R,2R′)-3,3′-disulfanediyl bis(2-acetamidopropanamide) (diNACA).

### BACKGROUND

According to the American Academy of Ophthalmology, AMD is defined by the presence of at least intermediate-size drusen (63 μm or larger in diameter), or yellowish accumulations of debris within Bruch's membrane; retinal pigment epithelium (RPE) abnormalities such as hypopigmentation or hyperpigmentation; reticular pseudodrusen or subretinal deposits; and/or the presence of any of the following features: advanced non-neovascular disease or geographic atrophy (GA), which is a circumscribed area or areas of RPE cell loss, and neovascular disease (exudative, wet) including choroidal neovascularization, polypoidal choroidal vasculopathy, or retinal angiomatous proliferation (Datta et al., 2017).

Age-related macular degeneration (AMD) is the term used for describing lost or blurred vision in the center of the visual field. A difficulty with detecting early stages of AMD is the lack of symptoms, which are very gradual and may affect one or both eyes. While all vision may not be lost, the loss of vision in the center of the visual field makes it difficult to recognize objects, drive, read, or perform normal activities. Typically, AMD is a disease that affects individuals later in life, and is made worse by smoking, hypertension, atherosclerosis, elevated cholesterol, obesity, fat intake, and exposure to sunlight. There are also genetic components to AMD, as sibling studies have shown an increase in recurrence ratios, and, to date, at least 5 different genes have shown some linkage to AMD. Unfortunately, AMD is a complex disease that results from a variety of environmental, genetic, and lifestyle factors.

Clinical signs of AMD include a distortion in the visual field, which typically takes the form of metamorphopsia, which is a type of vision distortion in which a grid of straight lines appears wavy and parts of the grid may appear blank. Other symptoms of AMD include: slow recovery of visual function as a result of exposure to bright light (e.g., using a photostress test), a drastic decrease in visual acuity, blurred vision, trouble discerning colors, and a loss of contrast sensitivity. AMD shows a processor accumulation of drusen deposits in the macula, between the retinal pigment epithelium, and the underlying choroid. These drusen are the build-up of extracellular proteins and lipids that are believed to damage the retina over time. However, the presence of drusen is not indicative of disease progression, as the majority of people over age 60 have drusen without any negative effects. Various stages of AMD are known, and are generally divided into early AMD, intermediate AMD, late AMD, Dry (or nonexudative) AMD, atrophic (or geographic) AMD (GA; GA may also be called GA-AMD), and/or wet (or exudative) AMD. FDA-approved drugs for wet AMD are shown in Table 1. FDA-approved drugs for wet AMD are shown in Table 1.

TABLE 1
FDA-approved Drugs for wet AMD.

| | | | | Typical |
|---|---|---|---|---|
| | Active | Mechanism | Delivery | Dosing |
| Tradename | ingredient | of Action | Route | Frequency |
| BEOVU | brolucizumab | VEGF inhibitor | Intravitreal | Every 8-12 |
| | | | injection | weeks |
| EYLEA | aflibercept | VEGF inhibitor | Intravitreal | Every 4-8 |
| | | | injection | weeks |
| EYLEA | aflibercept 8 | VEGF inhibitor | Intravitreal | Every 3-4 |
| HD | mg | | injection | months |
| LUCENTIS | ranibizumab | VEGF inhibitor | Intravitreal | Monthly or |
| | | | injection | as needed |
| SUSVIMO | ranibizumab | VEGF inhibitor | Refillable | Every 6 |
| | | | Intravitreal | months |
| | | | Implant | |
| VABYSMO | faricimab-svoa | VEGF and | Intravitreal | Every 1-4 |
| | | Ang-2 inhibitor | Injection | months |

*VEGF: Vascular Endothelial Growth Factor; Ang-2: Angiopoietin-2

FDA-approved drugs for GA are shown in Table 2.

TABLE 2
FDA-approved Drugs for GA

| | Active | | Delivery | Typical |
|---|---|---|---|---|
| Tradename | ingredient | Mechanism of Action | Route | Frequency |
| IZERVAY | Avacincaptad | Complement C5 | Intravitreal | Monthly |
| | pegol | inhibitor designed to | Injection | |
| | | target the source | | |
| | | of retinal cell death | | |
| SYFOVRE | Pegcetacoplan | Complement inhibitor | Intravitreal | Every 1-2 |
| | | that targets the C3 | Injection | months |
| | | protein to slow | | |
| | | the progression of | | |
| | | retinal cell damage | | |

Therapeutic approaches for AMD also include attempts to slow down the degenerative process by the use of vitamin and mineral supplements, however, these agents have not been demonstrated to be effective. All drugs in Tables 1 and 2 are intravitreal injections or implants. Currently, there is no FDA-approved oral therapy for any type of AMD that slows or stops the evolution of the disease or restores vision.

As such, there still exists a need for novel compositions and methods for treatment of age-related macular degeneration, including oral dosage forms.

### SUMMARY

As embodied and broadly described herein, an aspect of the present disclosure relates to a method for treatment of age-related macular degeneration, geographic atrophy, glaucoma or diabetic retinopathy in an animal or human in need thereof, the method comprising: providing an effective amount of an N-acetylcysteine amide (NACA) sufficient to inhibit degradation of vision. In one aspect, the eye disease is age-related macular degeneration. In another aspect, the eye disease is geographic atrophy. In another aspect, the eye disease is glaucoma. In another aspect, the eye disease is diabetic retinopathy. In another aspect, the NACA is provided orally, peritoneally, intravenously, dermally, bucally, sublingually, topically, topical ocularly, intraocularly, intravitreally, transmucosally, or by inhalation. In another aspect, the NACA inhibits the reduction in vision based on a primary endpoint, a secondary endpoint, or both. In another aspect, the NACA is at least one of: dosed as multiple tablets per day; dosed for at least 3 months, 6 months, or 12 months; dosed for 24 months; dosed for more than 24 months; dosed in one or more tablets, each containing 200, 225, 250, 275, 300, 350, 400, 450, 500, 600, 700, 800, 900, or 1,000 mg NACA, once daily; dosed in one or more tablets, each containing 200, 225, 250, 275, 300, 350, 400, 450, 500, 600, 700, 800, 900, or 1,000 mg NACA, twice daily; dosed in one or more tablets, each containing 200, 225, 250, 275, 300, 350, 400, 450, 500, 600, 700, 800, 900, or 1,000 mg NACA, thrice daily; dosed in one or more intravitreal implants, each containing 100, 200, 225, 250, 275, 300, 350, 400, 450, 500, 600, 700, 800, 900, or 1,000 μg diNACA, once every 3 months; dosed in one or more intravitreal implants, each containing 100, 200, 225, 250, 275, 300, 350, 400, 450, 500, 600, 700, 800, 900, or 1,000 μg diNACA, once every 6 months; or dosed in one or more intravitreal implants, each containing 100, 200, 225, 250, 275, 300, 350, 400, 450, 500, 600, 700, 800, 900, or 1,000 μg diNACA, once every year.

As embodied and broadly described herein, an aspect of the present disclosure relates to a method for treatment of age-related macular degeneration, geographic atrophy, glaucoma or diabetic retinopathy in an animal or human in need thereof, the method comprising: providing an effective amount of (2R,2R′)-3,3′-disulfanediyl bis(2-acetamidopropanamide) (diNACA) sufficient to inhibit degradation of vision. In one aspect, the eye disease is age-related macular degeneration. In another aspect, the eye disease is geographic atrophy. In another aspect, the eye disease is glaucoma. In another aspect, the eye disease is diabetic retinopathy. In another aspect, the diNACA is provided orally, peritoneally, intravenously, dermally, bucally, sublingually, topically, topical ocularly, intraocularly, intravitreally, transmucosally, or by inhalation. In another aspect, the diNACA inhibits the reduction in vision based on a primary endpoint. In another aspect, the diNACA inhibits the reduction in vision based on a secondary endpoint. In another aspect, the di NACA is at least one of: dosed as multiple tablets per day; dosed for at least 3 months, 6 months, or 12 months; dosed for 24 months; dosed for more than 24 months; dosed in one or more tablets, each containing 200, 225, 250, 275, 300, 350, 400, 450, 500, 600, 700, 800, 900, or 1,000 mg NACA, once daily; dosed in one or more tablets, each containing 200, 225, 250, 275, 300, 350, 400, 450, 500, 600, 700, 800, 900, or 1,000 mg NACA, twice daily; dosed in one or more tablets, each containing 200, 225, 250, 275, 300, 350, 400, 450, 500, 600, 700, 800, 900, or 1,000 mg NACA, thrice daily; dosed for at least 6 months, or 12 months; dosed for at least 24 months; dosed in one or more intravitreal implants, each containing 100, 200, 225, 250, 275, 300, 350, 400, 450, 500, 600, 700, 800, 900, or 1,000 μg diNACA, once monthly; or dosed in one or more intravitreal implants, each containing 100, 200, 225, 250, 275, 300, 350, 400, 450, 500, 600, 700, 800, 900, or 1,000 μg diNACA, once every 2 months. In another aspect, the diNACA is dosed in one or more intravitreal implants, each containing 100, 200, 225, 250, 275, 300, 350, 400, 450, 500, 600, 700, 800, 900, or 1,000 μg diNACA, once every 3 months. In another aspect, the diNACA is dosed in one or more intravitreal implants, each containing 100, 200, 225, 250, 275, 300, 350, 400, 450, 500, 600, 700, 800, 900, or 1,000 μg diNACA, once every 6 months. In another aspect, the diNACA is dosed in one or more intravitreal implants, each containing 100, 200, 225, 250, 275, 300, 350, 400, 450, 500, 600, 700, 800, 900, or 1,000 μg diNACA, once every year.

In accordance with an embodiment, the present disclosure provides a method for the treatment of age-related macular degeneration, glaucoma, and/or diabetic retinopathy in an animal or human that comprises administering to the animal or human a therapeutically effective amount of N-acetylcysteine amide (NACA) or (2R,2R′)-3,3′-disulfanediyl bis(2-acetamidopropanamide) (diNACA). In one aspect, the NACA is provided in or with a pharmaceutically acceptable carrier. In another aspect, the NACA is administered intraocularly, subretinally, intravitreally, orally, intravenously, intramuscularly, topically, sublingually, or rectally. In another aspect, the NACA is administered in daily doses of about 0.5, 1, 2, 3, 4, 5, 6, 7, 8, 9, 10, 15, 20, 25, 30, 40, 50, 60, 70, 75, 80, 90, 100, 110, 120, 125, 130, 140 or 150 mg/Kg. In another aspect, NACA is administered two or three times daily. In another aspect, NACA is administered with a second active agent selected from at least one of ascorbic acid, cysteine hydrochloride, sodium bisulfate, sodium metabisulfite, sodium sulfite, ascorbyl palmitate, butylated hydroxyanisole (BHA), butylated hydroxytouene (BHT), lecithin, propyl gallate, α-tocopherol, citric acid, ethylenediamine tetraacetic acid (EDTA), sorbitol, tartaric acid, or phosphoric acid. In another aspect, the dose for administration is 100, 150, 150, 300, 333, 400, 500, 600, 700, 750, 800, 900, 1,000, 2,500, 5,000, 7,500, or 10,000 mg per dose. In another aspect, the NACA dose for administration is 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per dose. In another aspect, the NACA is delivered orally via a mini-tablet, capsule, tablet, effervescent, dual release, mixed release, sachet, powder, or liquid. In another aspect, the NACA is administered prophylactically to prevent age-related macular degeneration, glaucoma, and/or diabetic retinopathy. In another aspect, the animal is a human. In another aspect, the dose for administration is between 100 and 900 micrograms per dose. In another aspect, the dose for administration is 100, 150, 150, 300, 350, 400, 500, 600, 700, 750, 800, 900, 1,000, 2,500, 5,000, 7,500, or 10,000 micrograms per dose. In another aspect, the diNACA is administered as an implant one, two, or three times per year.

In another aspect, diNACA is provided in or with a pharmaceutically acceptable carrier. In another aspect, diNACA is administered intraocularly, subretinally, intravitreally, orally, intravenously, intramuscularly, topically, sublingually, or rectally. In another aspect, the diNACA is administered in daily doses of about 0.5, 1, 2, 3, 4, 5, 6, 7, 8, 9, 10, 15, 20, 25, 30, 40, 50, 60, 70, 75, 80, 90, 100, 110, 120, 125, 130, 140 or 150 mg/Kg. In another aspect, diNACA is administered two or three times daily. In another aspect, diNACA is administered with a second active agent selected from at least one of ascorbic acid, cysteine hydrochloride, sodium bisulfate, sodium metabisulfite, sodium sulfite, ascorbyl palmitate, butylated hydroxyanisole (BHA), butylated hydroxytouene (BHT), lecithin, propyl gallate, α-tocopherol, citric acid, ethylenediamine tetraacetic acid (EDTA), sorbitol, tartaric acid, or phosphoric acid. In another aspect, the diNACA dose for administration is 100, 150, 150, 300, 333, 400, 500, 600, 700, 750, 800, 900, 1,000, 2,500, 5,000, 7,500, or 10,000 mg per dose. In another aspect, the diNACA dose for administration is 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per dose. In another aspect, diNACA is delivered orally via a mini-tablet, capsule, tablet, effervescent, dual release, mixed release, sachet, powder, or liquid. In another aspect, diNACA is administered prophylactically to prevent age-related macular degeneration, glaucoma, and/or diabetic retinopathy. In another aspect, the animal is a human. In another aspect, the dose for administration is between 100 and 900 micrograms per dose. In another aspect, the dose for administration is 100, 150, 150, 300, 350, 400, 500, 600, 700, 750, 800, 900, 1,000, 2,500, 5,000, 7,500, or 10,000 micrograms per dose. In another aspect, the diNACA is administered as an implant one, two, or three times per year.

### BRIEF DESCRIPTION OF THE DRAWINGS

For a more complete understanding of the features and advantages of the present disclosure, reference is now made to the detailed description of the disclosure along with the accompanying figures and in which:

FIG. 1 is a graph that shows the concentration of NACA in plasma.

FIG. 2 is a graph that shows the concentration of NACA in the aqueous humor.

FIG. 3 is a graph that shows the concentration of NACA in the vitreous humor.

FIG. 4 is a graph that shows the concentration of NACA in the retina.

FIG. 5 is a graph that shows a comparison of NACA:NAC Levels in the plasma and retina.

FIG. 6 shows the thioether derivatives of NAC and NACA for LCMS analyses.

FIG. 7 shows significant slowing of photoreceptor cell loss with NPI-011 (NCT04355689) (blue/solid=active; red/dashed=placebo) (P-values presented are two-sample t-tests to assess change from baseline treatment differences at each visit).

FIG. 8 shows pharmacokinetics of NACA (NPI-001) orally dosed over 14 days in subjects 18 years and older.

### DETAILED DESCRIPTION

While the making and using of various aspects of the present disclosure are discussed in detail below, it should be appreciated that the present disclosure provides many applicable inventive concepts that can be embodied in a wide variety of specific contexts. The specific aspects discussed herein are merely illustrative of specific ways to make and use the disclosure and do not delimit the scope of the disclosure.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific aspects of the disclosure, but their usage does not delimit the disclosure, except as outlined in the claims.

Age-related macular degeneration (“AMD”) comprises a large group of inherited vision disorders that cause progressive loss of photoreceptor cells of the retina, leading to severe vision impairment and often incurable blindness. The most common form of AMD is a rod-cone dystrophy, in which the first symptom is night blindness, followed by progressive loss in the peripheral visual field in daylight, and eventually leading to blindness after several decades. As a common pathology, rod photoreceptors die early, whereas light-insensitive, morphologically altered cone photoreceptors persist longer. Various stages of AMD are known, and are generally divided into early AMD, intermediate AMD, late AMD, Dry (or nonexudative) AMD, atrophic (or geographic) AMD (GA; GA may also be called GA-AMD), and/or wet (or exudative) AMD.

Diabetic retinopathy (DR), sometimes referred to as diabetic eye disease, in which diabetes mellitus leads to damage to the retina, and is a leading cause of blindness. Typically, DR affects up to 80 percent of diabetic patients. Importantly, the longer a patient has diabetes, the higher the chances of developing diabetic retinopathy. In the United States, diabetic retinopathy accounts for 12% of all new cases of blindness, and is the leading cause of blindness in patients aged 20 to 64.

Glaucoma described several eye diseases that result from damage to the optic nerve leading to loss of vision. Typical symptoms of glaucoma include, e.g., eye pain, blurred vision, mid-dilated pupil, redness of the eye, and nausea. An increase in intraocular pressure is a major risk factor for glaucoma, as are a family history of glaucoma and high blood pressure, however, the etiology of glaucoma is still under investigation.

In accordance with another embodiment, the present disclosure includes a method for the treatment of age-related macular degeneration, glaucoma, and/or diabetic retinopathy comprising: identifying a human in need of treatment for age-related macular degeneration, glaucoma, and/or diabetic retinopathy; and administering to the human a therapeutically effective amount of N-acetylcysteine amide (NACA) or (2R,2R′)-3,3′-disulfanediyl bis(2-acetamidopropanamide) (diNACA) sufficient to treat age-related macular degeneration, glaucoma, and/or diabetic retinopathy.

Oxidative stress is common to all three non-genetic risk factors of AMD-aging, smoking, and high-fat diet (Datta et al., 2017). AMD disease processes are thought to be triggered when the redox status of RPE cells switches from an anti-oxidant stage to a pro-oxidant stage. Damaged mitochondria are a source of reactive oxygen species (ROS), if not removed by mitophagy and has been shown to be associated with AMD (Karunadharam et al., 2010). Mitophagy can be analyzed by measuring the expression of mitochondrial proteins, quantifying the levels of mitochondrial DNA, measuring the activity of enzyme Citrate Synthetase, and by electron microscopy (Williams et al., 2017). Downregulation of mitophagy and mitochondrial activity has been noted in AMD patient derived iPSC-RPE cells (Golestaneh et al., 2016). Another mechanism regulating cellular redox homeostasis is regulated by transcription factor NRF2 that is upstream of genes involved in managing cellular oxidative stress response (see Datta et al 2017 for more details). It directly regulates the expression of several key oxidative stress pathway genes, such as Catalase and SOD.

In addition, NRF2 has been shown to regulate levels of cellular anti-oxidants glutathione and thioredoxin. NRF2 expression is shown to decrease with aging (Suzuki et al., 2008) and after acute oxidative stress in RPE cells (Sachdeva et al., 2014). Measurement of the expression of NRF2 target genes, and the levels of glutathione and thioredoxin provides a critical readout of cellular redox status and is directly associated with AMD pathogenesis.

Oxidative stress targets that rejuvenate endogenous intracellular antioxidant systems have conceptual merit because the oxidative stress can be neutralized within cells where ROS are generated (Datta et al., 2017).

Therefore, treatment with an antioxidant should slow or stop development of GA-AMD. It is known that patients with retinitis pigmentosa (RP) suffer cone cell loss due to oxidative stress in the retina (Komeima et al., 2006; Komeima et al., 2007). The FIGHT RP trial demonstrated improvement in cone function in patients with RP during a 24-week treatment period with NAC. This suggests that some of the visual dysfunction is due to cones that are functioning suboptimally due to oxidative stress. It is reasonable to hypothesize that long term reduction of oxidative stress with oral NAC could promote survival of cones and thereby stop or slow the inexorable reduction in visual fields that leads to blindness. A large phase III, multicenter, placebo-controlled, double masked clinical trial is in progress to test this hypothesis (Raghu et al., 2021).

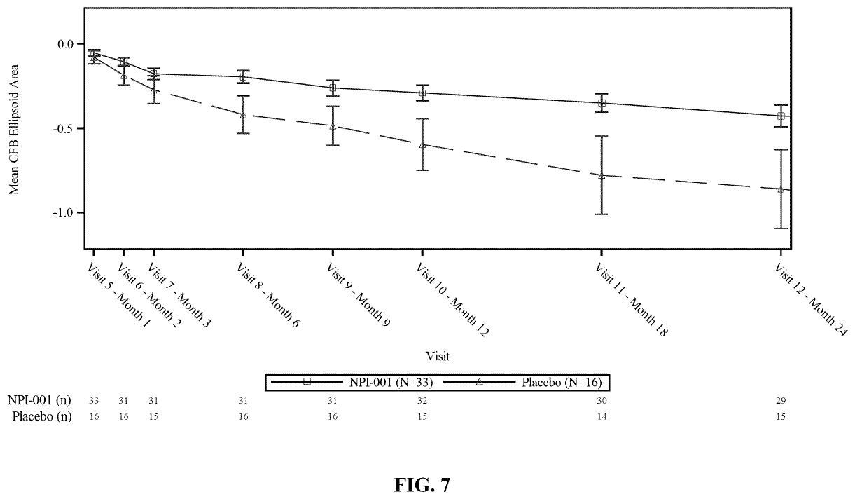

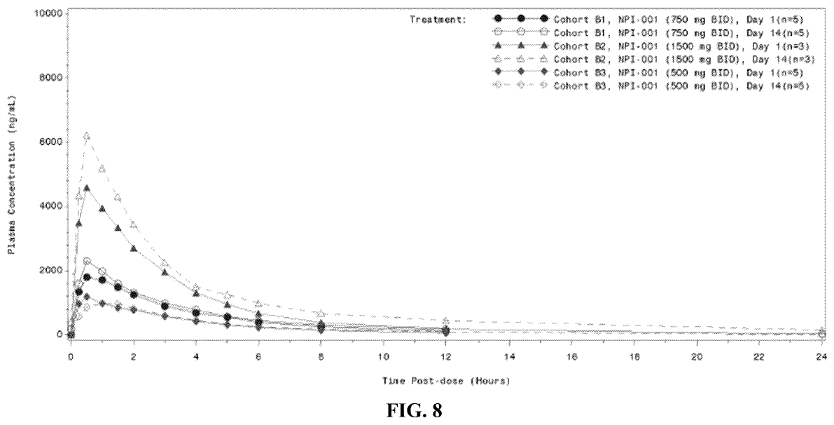

One drug that shows promise is N-acetylcysteine amide (NACA, NPI-001) because it is the first drug that has ever been demonstrated efficacious in slowing photoreceptor cell loss in patients with RP, a disease involving oxidative stress. Oral NPI-001 tablets (250 mg, BID) significantly delayed progression of RP measured by photoreceptor cell preservation in participants with USH over 24 months compared with placebo tablets (FIG. 7) (Kern and Wall, 2026).

DiNACA is the dimer of NACA. DiNACA, NACA and NAC were observed in rat plasma following oral gavage of diNACA 200 milligrams per kilogram in rat. This study demonstrated that diNACA serves as a prodrug to NACA and NAC, three antioxidative moieties (Reference: NACU1030 patent application).

N-acetyl-L-cysteine amide (NACA), also known as (R)-2-(acetylamino)-3-mercapto-propanamide, N-acetyl-L-cysteinamide, or acetylcysteinamide, has the structure:

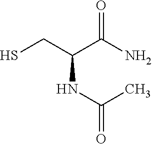

(2R,2R′)-3,3′-disulfanediyl bis(2-acetamidopropanamide) (diNACA), has the structure:

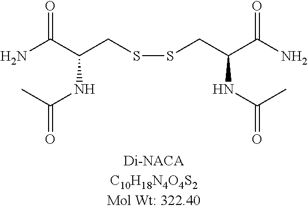 [SMILES: CC(=O)N[C@@H](CSSC[C@H](NC(C)=O)C(N)=O)C(N)=O]

(2R,2R′)-3,3′-disulfanediyl bis(2-acetamidopropanamide) (diNACA) is the dimer form of NACA. NACA or (2R,2R′)-3,3′-disulfanediyl bis(2-acetamidopropanamide) (diNACA) can be used in the treatment of AMD and/or DR.

Gluthathione (GSH) is a tripeptide, c-L-glutamyl-L-cysteinyl-glycine, found in all mammalian tissues. It has several important functions including detoxification of electrophiles, scavenging ROS<maintaining the thiol status of proteins, and regeneration of the reduced forms of vitamins C and E. GSH is the dominant non-protein thiol in mammalian cells; as such it is essential in maintaining the intracellular redox balance and the essential thiol status of proteins. Also, it is necessary for the function of some antioxidant enzymes such as the glutathione peroxidases.

Intracellular GSH levels are determined by the balance between production and loss. Production results from de novo synthesis and regeneration of GSH from GSSG by GSSG reductase. Generally, there is sufficient capacity in the GSSG reductase system to maintain all intracellular GSH in the reduced state, so little can be gained by ramping up that pathway. The major source of loss of intracellular GSH is transport out of cells. Intracellular GSH levels range from 1-8 mM while extracellular levels are only a few μM; this large concentration gradient essentially precludes transport of GSH into cells and once it is transported out of cells, it is rapidly degraded by γ-glutamyltranspeptidase. Inhibition of GSH transporters could theoretically increase intracellular GSH levels, but is potentially problematic because the transporters are not specific for GSH and their suppression could lead imbalance of other amino acids and peptides. Thus, intracellular GSH levels are modulated primarily by changes in synthesis.

GSH is synthesized in the cytosol of virtually all cells by two ATP-requiring enzymatic steps: L-glutamate+L-cysteine+ATP [→] γ-glutamyl-L-cysteine+ADP+Pi and γ-glutamyl-L-cysteine+L-glycine+ATP [→] GSH+ADP+Pi. The first reaction is rate-limiting and is catalyzed by glutamate cysteine ligase (GCL, EC 6.3.2.2). GCL is composed of a 73 Kd heavy catalytic subunit (GCLC) and a 30 Kd modifier subunit (GCLM), which are encoded by different genes. GCCL is regulated by nonallosteric competitive inhibition of GSH (Ki=2.3 mM) and by the availability of L-cysteine. The apparent Km of GLC for glutamate is 1.8 mM and intracellular glutamate concentration is roughly 10-fold higher so that glutamate is not limiting, but the Km for cysteine is 0.1-0.3 mM, which approximates its intracellular concentration. The second reaction is catalyzed by GSH synthase (GS, EC 6.3.2.3), which is 118 Kd and composed of two identical subunits. While GS is not felt to be important in regulation of GSH synthesis under normal conditions, it may play a role under stressful conditions because in response to surgical trauma, GSH levels and GS activity were reduced while GCL activity was unchanged. Furthermore, compared to increased expression of GCLC alone, increased expression of both GCLC and GS resulted in higher levels of GSH. In order to maximize the effects of increasing synthetic enzymes, it is necessary to provide increased levels of cysteine. In cultured neurons, 90% of cysteine uptake occurs through by the sodium-dependent excitatory amino acid transporter (EAAT) system. There are five EAATs and cysteine uptake by neurons occurs predominantly by EAAT3 more commonly known as excitatory amino acid carrier-1 (EAAC1). Under normal circumstances most EAAC1 is in the ER and only translocates to the plasma membrane when activated. This translocation is negatively regulated by glutamate transporter associated protein 3-18 (GTRAP3-18) and suppression of GTRAP3-18) increased GSH levels in neurons. Thus, internalization of cysteine provides a roadblock for GSH synthesis, but fortunately it can be bypassed by N-acetylcysteine (NAC) which readily enters cells even in the absence of activated EAAC1. Systemically administered NAC gains access to the CNS, increases GSH levels, and provides benefits in neurodegenerative disorders in which oxidative stress is an important part of the pathogenesis.

All cellular compartments must be protected against oxidative damage, including the cytoplasm, mitochondria, and the nucleus. The present inventors have previously performed gene transfer of enzymes that detoxify reactive oxygen species, but that approach requires the expression of two enzymes in the cytoplasm and two enzymes in mitochondria. In contrast, the present disclosure provides for protection of all cellular compartments with expression of only two enzymes in the cytosol because GSH is able to diffuse everywhere throughout cells.

NAC is used for the treatment of acetaminophen overdose at a dose of 140 mg/kg as the loading dose, followed by 70 mg/kg every 4 hours for 17 doses, starting 4 hours after the loading dose. In clinical studies, NAC has been administered orally from 400 to 1000 mg once daily and from 200 to 600 mg three times daily. However, following an oral dose of 600 mg in humans, NAC is rapidly absorbed and then rapidly cleared. The plasma half-life of NAC has been reported to be 2.5 hours and no NAC is detectable 10-12 hours after administration. During absorption, NAC is rapidly metabolized to cysteine, which is a direct precursor of glutathione. Based on this evidence, including that NACA is a precursor and/or carrier for NAC, it was expected that NACA would act similarly to NAC in vivo. However, the present inventors demonstrate that NACA acts very differently from NAC for the treatment of AMD.

N-Acetylcysteine Amide (NACA). Orally administered N-Acetylcysteine amide (NACA, NPI-001) has been found to be a particularly effective antioxidant to treat retinitis pigmentosa (See U.S. patent application Ser. No. 15/523,665). The NACA can be dosed at 200, 225, 250, 275, 300, 350, 400, 450, 500, 600, 700, 800, 900, or 1,000 per day, the NACA is dosed as multiple tablets per day. The NACA can be dosed as two tablets, each containing 200, 225, 250, 275, 300, 350, 400, 450, 500, 600, 700, 800, 900, or 1,000 mg NACA, once daily. The NACA can be dosed as two tablets, each containing 200, 225, 250, 275, 300, 350, 400, 450, 500, 600, 700, 800, 900, or 1,000 mg NACA, twice daily. The NACA can be dosed as two tablets, each containing 200, 225, 250, 275, 300, 350, 400, 450, 500, 600, 700, 800, 900, or 1,000 mg NACA, thrice daily. The NACA can be dosed for at least 6 months, or 12 months. The NACA can be dosed for at least 24 months. The NACA can be dosed for more than 24 months. The NACA can be dosed such that the NACA does not reach an appreciable daily steady-state concentration.

As used herein, the terms “effective amount” or “effective doses” refer to that amount of an agent to product the intended pharmacological, therapeutic or preventive results. The pharmacologically effective amount results in the amelioration of one or more signs or symptoms of a disease or condition or the advancement of a disease or conditions, or causes the regression of the disease or condition. For example, a therapeutically effective amount preferably refers to the amount of a therapeutic agent that decreases vision loss, the loss of overall visual acuity, the loss of visual field, by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an untreated control subject over a defined period of time, e.g., 2 weeks, one month, 2 months, 3 months, 6 months, one year, 2 years, 5 years, or longer. More than one dose may be required to provide an effective dose.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such as treatment may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater liver toxicity.

Thus, in connection with the administration of a drug, a drug which is “effective against” a disease or condition indicates that administration in a clinically appropriate manner results in a beneficial effect for at least a statistically significant fraction of patients, such as an improvement of symptoms, a cure, a reduction in disease signs or symptoms, extension of life, improvement in quality of life, or other effect generally recognized as positive by medical doctors familiar with treating the particular type of disease or condition.

As used herein, the term phrase “pharmaceutically acceptable carrier” is art recognized and includes a pharmaceutically acceptable material, composition or vehicle, suitable for administering compounds of the present disclosure to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. For example, pharmaceutically acceptable carriers for administration of cells typically is a carrier acceptable for delivery by injection, and do not include agents such as detergents or other compounds that could damage the cells to be delivered. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations, particularly phosphate buffered saline solutions which are preferred for intraocular delivery.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present disclosure include those suitable for oral, nasal, topical, transdermal, buccal, sublingual, intramuscular, intraperotineal, intraocular, intravitreal, subretinal, and/or other routes of parenteral administration. The specific route of administration will depend, inter alia, on the specific cell to be targeted. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

As used herein, the term “subject” refers to living organisms, in particular, humans. In certain embodiments, the living organism is an animal, in certain preferred embodiments, the subject is a mammal, in certain embodiments, the subject is a domesticated mammal or a primate including a non-human primate. Examples of subject include humans, monkeys, dogs, cats, mice, rates, cows, horses, goats, and sheep. A human subject may also be referred to as a subject or patient.

As used herein, the term “therapeutically effective amount,” refers to an amount of an agent which is effective, upon single or multiple does administration to the cell or subject, in prolonging the survivability of the patient with such a disorder, reducing one or more signs or symptoms of the disorder, preventing or delaying and the like beyond that expected in the absence of such treatment.

An agent or other therapeutic intervention can be administered to a subject, either alone or in combination with one or more additional therapeutic agents or interventions, as a pharmaceutical composition in mixture with conventional excipient, e.g., pharmaceutically acceptable carrier, or therapeutic treatments.

The pharmaceutical agents may be conveniently administered in unit dosage form and may be prepared by any of the methods well known in the pharmaceutical arts, e.g., as described in Remington's Pharmaceutical Sciences (Mack Pub. Co., Easton, PA, 1985). Formulations for parenteral administration may contain as common excipients such as sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of certain agents.

The present disclosure is directed to the use of NACA to prevent and/or treat UARP. In one embodiment, the present disclosure includes a method for use of N-acetylcysteine amide (NACA) or (2R,2R′)-3,3′-disulfanediyl bis(2-acetamidopropanamide) (diNACA) for the prevention and/or treatment of UARP in a human that comprises administering to the human therapeutically effective amount of NACA. In some embodiments, the NACA is provided in or with a pharmaceutically acceptable carrier. In other embodiments, the NACA is administered intraocularly, subretinally, intravitreally, orally, intravenously, intramuscularly, topically, sublingually, or rectally.

It will be appreciated that the actual preferred amounts of active compounds used in a given therapy will vary according to e.g., the specific compound being utilized, the particular composition formulated, the mode of administration and characteristics of the subject, e.g., the species, sex, weight, general health and age of the subject. Optimal administration rates for a given protocol of administration can be readily ascertained by those skilled in the art using conventional dosage determination tests conducted with regard to the forgoing guidelines.

Ranges provided herein are understood to be shorthand for all of the values within the range in increments of one percent, five percent, and ten percent.

As used herein, the aspects of this disclosure are defined to include pharmaceutically acceptable derivatives thereof. A “pharmaceutically acceptable derivative” means any pharmaceutically salt, ester, salt of an ester, or other derivative of a compound of this disclosure which, upon administration to a recipient, is capable of providing (directly or indirectly) a compound of this disclosure. Particularly favored derivatives are those that increase the bioavailability of the compounds of this disclosure when such compounds are administered to a mammal (e.g., by allowing an orally administered compound to be more readily absorbed into the blood, to increase serum stability or decrease clearance rate of the compound) or which enhance delivery of the parent compound to a biological compartment (e.g., the brain or lymphatic system) relative to the parent species. Derivatives include derivatives where a group which enhances aqueous solubility or active transport through the gut membrane is appended to the structure of formulae described herein.

The embodiments of this disclosure may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion. Pharmaceutically acceptable salts of the compounds of this disclosure include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-napthalenesulfonate, nicotinate, nitrate, palmoate, phosphate, picrate, pivalate, propionate, salicylate, succinate, sulfate, tartrate, tosylate, and undeconaoate. Salts derived from appropriate bases include alkali metal (e.g., sodium), alkaline earth metal (e.g., magnesium), ammonium and N-(alkyl) 4+ salts. This disclosure also envisions the quaternization of any basic nitrogen-containing groups of the compounds disclosed herein. Water or oil-soluble or dispersible products may be obtained by such quaternization.

The embodiments of the disclosure can, for example, be administered by injection, intraocularly, intravitreally, subretinal, intravenously, intraarterially, subdermally, intramuscularly, or subcutaneously; or orally, buccally, nasally, transmucosally, directly to a diseased organ by catheter, topically, or in an ophthalmic preparation, with a dosage ranging from about 0.001 to about 100 mg/kg of body weight, or according to the requirements of the particular drug and more preferably from 0.5-10 mg/kg of body weight. It is understood that when a compound is delivered directly to the eye, considerations such as body weight have less bearing on the dose.

Frequency of dosing will depend on the agent administered, the progression of the disease or condition in the subject, and other considerations known to those of skill in the art. For example, pharmacokinetic and pharmacodynamics considerations for compositions delivered to the eye, or even compartments within the eye, are different, e.g., clearance in the subretinal space is very low. Therefore, dosing can be as infrequent as once a month, once every three months, once every six months, once a year, once every five years, or less. If systemic administration of antioxidants is to be performed in conjunction with administration of expression constructs to the subretinal space, it is expected that the dosing frequency of the antioxidant will be higher than the expression construct, e.g., one or more times daily, one or more times weekly.

Dosing may be determined in conjunction with monitoring of one or more signs or symptoms of the disease, e.g., visual acuity, visual field, night visions, etc. The amount of active ingredient that may be combined with the carrier materials to produce a single dosage form will vary depending upon the host treated and the particular mode of administration. A typical preparation will contain from about 1% to about 95% active compound (w/w). Alternatively, such preparations contain from about 20% to about 80% active compound. Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity ad course of the disease, condition or symptoms, the patient's disposition to the disease, condition or symptoms and the judgment of the treating physician.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, TWEEN® 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as TWEENs® or SPAN® and/or other similar emulsifying agents or bioavailability enhancers which are commonly used in the manufacture of pharmaceutically acceptable solid, liquid, or other dosage forms may also be used for the purposes of formulation.

In one or more embodiments, NACA or diNACA is administered in daily doses of about 0.5, 1, 2, 3, 4, 5, 6, 7, 8, 9, 10, 15, 20, 25, 30, 40, 50, 60, 75, 80, 90, 100, 110, 120, 125, 130, 140, or 150 mg/Kg. In other embodiments, NACA or diNACA is administered two or three times daily. In another aspect, NACA or diNACA is administered with a second active agent selected from ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

In some embodiments, the dose of NACA or diNACA for administration is, 100, 150, 150, 300, 333, 400, 500, 600, 700, 750, 800, 900, 1,000, 2,500, 5,000, 7,500, or 10,000 mg per dose. In another aspect, the dose for administration is 0.1-0.25, 0.1-0.4, 0.35-0.5, 0.5-1, 102, 1-3, 1-4, 1-5, 1-2.5, 2.5-3.5, 4-6, 5-8, 6-9, 7-10 grams per dose. In another aspect, the NACA or diNACA is delivered orally via a mini-tablet, capsule, tablet, effervescent, dual release, mixed release, sachet, powder, or liquid. In another aspect, the NACA is administered prophylactically to prevent and/or treat UARP.

In accordance with an embodiment, the present disclosure provides a method for the prevention, amelioration, or treatment of a disease or condition associated with oxidative stress in a subject comprising administration of a therapeutically effective amount of NACA, to increase the amount of glutathione expressed in the tissues of the subject.

As used herein, “active oxygen species” or “reactive oxygen species” are understood as transfer of one or two electrons produces superoxide, an anion with the form 02″, or peroxide anions, having the formula O2-″ or compounds containing an O—O single bond, for example hydrogen peroxides and lipid peroxides. Such superoxides and peroxides are highly reactive and can cause damage to cellular components including proteins, nucleic acids, and lipids.

As used herein, the term “agent” refers to a therapeutically active compounds or a potentially therapeutic active compound, e.g., an antioxidant. An agent can be a previously known or unknown compound. As used herein, an agent is typically a non-cell based compound, however, an agent can include a biological therapeutic agent, e.g., peptide or nucleic acid therapeutic, e.g., siRNA, shRNA, cytokine, antibody, etc.

As used herein, the terms “amelioration” or “treatment” is understood as meaning to lessen or decrease at least one sign, symptom, indication, or effect of a specific disease or condition. For example, amelioration or treatment of age-related macular degeneration (AMD) can be to reduce, delay, or eliminate one or more signs or symptoms of AMD including, but not limited to, a reduction in night vision, a reduction in overall visual acuity, a reduction in visual field, a reduction in the cone density in one or more quadrants of the retina, thinning of retina, particularly the outer nuclear layer, reduction in a- or b-wave amplitudes on scotopic or photopic electroretinograms (ERGs); or any other clinically acceptable indicators of disease state or progression. Amelioration and treatment can require the administration of more than one dose of an agent, either alone or in conduction with other therapeutic agents and interventions. Amelioration or treatment does not require that the disease or condition be cured.

As used herein, the term “antioxidant” refers to a molecule for slowing or preventing the oxidation of other molecules. Oxidation is a chemical reaction that transfers electrons from a substance to an oxidizing agent. Such reactions can be promoted by or produce superoxide anions or peroxides. Oxidation reactions can produce free radicals, which start chain reaction that damage cells. Antioxidants terminate these chain reactions by removing free radical intermediates, and inhibit other oxidation reactions by being oxidized themselves. As a result, antioxidants are often reducing agents such as thiols, ascorbic acid or polyphenols. Antioxidants include, but are not limited to, α-tocopherol, ascorbic acid, Mn(III)tetrakis (4-benzoic acid) porphyrin, α-lipoic acid, and n-acetylcysteine.

As used herein, the term “co-administration” refers to the administration of one or more agents to a subject such that the agents are present and active in the subject at the same time. Co-administration does not require a preparation of an admixture of the agents or simultaneous administration of the agents.

As used herein, the terms “effective amount” or “effective doses” refer to that amount of an agent to product the intended pharmacological, therapeutic or preventive results. The pharmacologically effective amount results in the amelioration of one or more signs or symptoms of a disease or condition or the advancement of a disease or conditions, or causes the regression of the disease or condition. For example, a therapeutically effective amount preferably refers to the amount of a therapeutic agent that decreases vision loss, the loss of overall visual acuity, the loss of visual field, by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an untreated control subject over a defined period of time, e.g., 2 weeks, one month, 2 months, 3 months, 6 months, one year, 2 years, 5 years, or longer. More than one dose may be required to provide an effective dose.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such as treatment may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater liver toxicity.

Thus, in connection with the administration of a drug, a drug which is “effective against” a disease or condition indicates that administration in a clinically appropriate manner results in a beneficial effect for at least a statistically significant fraction of patients, such as an improvement of symptoms, a cure, a reduction in disease signs or symptoms, extension of life, improvement in quality of life, or other effect generally recognized as positive by medical doctors familiar with treating the particular type of disease or condition.

As used herein, the phrase “oxidative stress related ocular disorders” includes, but is not limited to, age-related macular degeneration, macular degeneration including age related macular degeneration (AMD) both wet and dry, diabetic retinopathy, Lebers optic neuropathy, and optic neuritis.

As used herein, the terms “peroxidases” or “a peroxide metabolizing enzyme” refer to a large family of enzymes that typically catalyze a reaction of the form:

ROOR1+electron donor (2 e−)+2H+→ROH+R1OH For many of these enzymes the optimal substrate is hydrogen peroxide, wherein each R is H, but others are more active with organic hydroperoxides such as lipid peroxides. Peroxidases can contain a heme cofactor in their active sites, or redox-active cysteine or selenocysteine residues.

As used herein, the term phrase “pharmaceutically acceptable carrier” is art recognized and includes a pharmaceutically acceptable material, composition or vehicle, suitable for administering compounds of the present disclosure to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. For example, pharmaceutically acceptable carriers for administration of cells typically is a carrier acceptable for delivery by injection, and do not include agents such as detergents or other compounds that could damage the cells to be delivered. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations, particularly phosphate buffered saline solutions which are preferred for intraocular delivery.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present disclosure include those suitable for oral, nasal, topical, transdermal, buccal, sublingual, intramuscular, intraperotineal, intraocular, intravitreal, subretinal, and/or other routes of parenteral administration. The specific route of administration will depend, inter alia, on the specific cell to be targeted. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

As used herein, “plurality” is understood to mean more than one. For example, a plurality refers to at least two, three, four, five, or more.

As used herein, the term a “polypeptide” or “peptide” is understood as two or more independently selected natural or non-natural amino acids joined by a covalent bond (e.g., a peptide bond). A peptide can include 2, 3, 4, 5, 6, 7, 8, 9, 10, 11, 12, 13, 14, 15, 16, 17, 18, 19, 20, or more natural or non-natural amino acids joined by peptide bonds. Polypeptides as described herein include full-length proteins (e.g., fully processed proteins) as well as shorter amino acids sequences (e.g., fragments of naturally occurring proteins or synthetic polypeptide fragments).

As used herein, “prevention” is understood as to limit, reduce the rate or degree of onset, or inhibit the development of at least one sign or symptom of a disease or condition particularly in a subject prone to developing the disease or disorder. For example, a subject having a mutation in a gene, such as the opsin gene, is likely to develop AMD. The age of onset of one or more symptoms of the disease can sometimes be determined by the specific mutation. Prevention can include the delay of onset of one or more signs or symptoms of AMD and need not be prevention of appearance of at least one sign or symptom of the disease throughout the lifetime of the subject. Prevention can require the administration of more than one does of an agent or therapeutic.

As used herein, the term “small molecule” refers to a compound, typically an organic compound, having a molecular weight of no more than about 1500 Da, 1000 Da, 750 Da, or 500 Da. In an embodiment, a small molecule does not include a polypeptide or nucleic acid including only natural amino acids and/or nucleotides.

As used herein, the term “subject” refers to living organisms, in particular, humans. In certain embodiments, the living organism is an animal, in certain preferred embodiments, the subject is a mammal, in certain embodiments, the subject is a domesticated mammal or a primate including a non-human primate. Examples of subject include humans, monkeys, dogs, cats, mice, rates, cows, horses, goats, and sheep. A human subject may also be referred to as a subject or patient.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition or syndrome. Methods for identification of subjects suffering from or suspected of suffering from conditions such as AMD and age-related macular degeneration (AMD) is within the ability of those in the art. Subjects suffering from, and suspected of suffering from, a specific disease, condition, or syndrome are not necessarily two distinct groups.

As used herein, “superoxide dismutase” is understood as an enzyme that dismutation of superoxide into oxygen and hydrogen peroxide. Examples include, but are not limited to SOD1, SOD2, and SOD3. Sod1 and SOD3 are two isoforms of Cu—Zn-containing superoxide dismutase enzymes exists in mammals. Cu—Zn-SOD or SOD1, is found in the intracellular space, and extracellular SOD (ECSOD or SOD3) predominantly is found in the extracellular matrix of most tissues.

As used herein, the term “therapeutically effective amount,” refers to an amount of an agent which is effective, upon single or multiple does administration to the cell or subject, in prolonging the survivability of the patient with such a disorder, reducing one or more signs or symptoms of the disorder, preventing or delaying and the like beyond that expected in the absence of such treatment.

An agent or other therapeutic intervention can be administered to a subject, either alone or in combination with one or more additional therapeutic agents or interventions, as a pharmaceutical composition in mixture with conventional excipient, e.g., pharmaceutically acceptable carrier, or therapeutic treatments.

The pharmaceutical agents may be conveniently administered in unit dosage form and may be prepared by any of the methods well known in the pharmaceutical arts, e.g., as described in Remington's Pharmaceutical Sciences (Mack Pub. Co., Easton, PA, 1985). Formulations for parenteral administration may contain as common excipients such as sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of certain agents.

The present disclosure is directed to the use of NACA to treat AMD. In one embodiment, the present disclosure includes a method for the treatment of age-related macular degeneration in a human that comprises administering to the human therapeutically effective amount of NACA. In some embodiments, the NACA is provided in or with a pharmaceutically acceptable carrier. In other embodiments, the NACA is administered intraocularly, subretinally, intravitreally, orally, intravenously, intramuscularly, topically, sublingually, or rectally.

Animals and Treatments. Mice were treated in accordance with the recommendations of the Association for Research in Vision and Ophthalmology. Litters of homozygous rd10/rd10 mice (B6.CXB1-Pde6brd10/J) and wild type C57/BL6 mice (The Jackson Laboratory, Bar Harbor, ME) were used for these studies.

Starting on post-natal day 28 (P28), animals were given normal drinking water (n=10 for each strain) or water containing 20 mg/ml NACA (n=10 for each strain).

Analysis of NACA and NAC. Upon sacrifice of the mice on P35, retina, aqueous, vitreous and plasma were collected, weighed, and stabilized as quickly as possible and stored at −80° C. prior to shipment to AIT Bioscience, LLC (Indianapolis, IN). The bioanalytical method (BAM.0445.01) for the quantitation of total NAC NACA in K2EDTA was based on derivatization, protein precipitation extraction, and LC-MS/MS instrumental analysis. Any disulfides were reduced to free thiols and subsequently reacted with 2-chloro-1-methylpyridinium iodide (CMPI) to form stable thioethers. The thioether derivatives were detected by LC-MS/MS. Stable label isotope internal standards were used. The method covered a concentration range from 50.0 to 50,000 ng/mL. The thiol moiety of NAC, NACA, and their respective internal standards oxidizes quickly in plasma through formation of disulfides. In order to determine total NAC and total NACA levels in plasma, tris(2-carboxyethyl)phosphine (TCEP) is added during the extraction to reduce disulfide bonds. Ammonium bicarbonate is added to control sample pH near neutral, as TCEP will otherwise acidify the aliquoted samples and hinder derivatization. The free analyte is then derivatized to a stable thioether using 2-chloro-1-methylpyridinium iodide (CMPI). N-acetyl-L-cysteine is used as the reference standard. The assay would not discern the enantiomer, N-acetyl-D-cysteine, if present. A sample volume of 25.0 μL was aliquotted into a 1.2 mL 96-well plate to which was added, in sequence, 25.0 μL internal standard solution (1000 ng/mL NAC-D3 and 1000 ng/mL NACA-D3 in water), 50.0 μL of ammonium bicarbonate (100 mM), 5.0 μL CMPI (60 mM in water), and 5.0 μL of TCEP (60 mM in water). Samples were allowed to react for 30 minutes. To precipitate proteins, 500 μL of acetonitrile was then added to all samples. The plate was covered and the mixtures were shaken and centrifuged. A 50.0 μL aliquot of the supernatant was transferred from each well to a clean plate containing 400 μL of water-acetonitrile (25-75) in each well, and mixed well prior to LC-MS injection.

FIG. 1 shows the thioether derivatives of NAC and NACA for LCMS analyses.

Samples were analyzed on a Waters Acquity liquid chromatograph interfaced with a Thermo Scientific TSQ Vantage triple quadrupole mass spectrometer with ESI ionization. Each extracted sample was injected (5.0 μL) onto a Waters BEH HILIC column (2.1×100 mm; 1.7 μm) equilibrated at 35° C. Mobile Phase A was ammonium formate (25 mM, pH 3.8). Mobile Phase B was acetonitrile. The LC gradient is tabled below:

| | Flow Rate | | |
|---|---|---|---|
| Time (min) | (mL/min) | % MP A | % MP B |
| 0.00 | 0.500 | 25.0 | 75.0 |
| 2.30 | 0.500 | 25.0 | 75.0 |

The retention time, mass transition and precursor charge state for each compound are as follows. The masses below are for the CMPI thioether derivatives.

| | Expected | | Product | Charge |
|---|---|---|---|---|
| | Retention | Precursor | Observed | State of |
| | Time | Mass/Charge | Mass/Charge | Precursor |
| Compound | (min) | (m/z) | (m/z) | Ion |
| N-Acetyl-L- | 1.90 | 255.080 | 126.16 | +1 |
| Cysteine (NAC) | | | | |
| N-Acetylcysteine | 1.25 | 254.096 | 126.16 | +1 |
| amide (NACA) | | | | |
| N-Acetyl-L-Cys-D₃ | 1.90 | 258.099 | 126.15 | +1 |
| N-Acetyl-L- | 1.25 | 257.115 | 126.15 | +1 |
| Cysteine-D₃ | | | | |

Peak area ratios from the calibration standard responses were regressed using a (1/concentration2) linear fit for N-Acetyl-L-Cysteine and N-Acetylcysteine amide.

NACA Preclinical Study Proposal for Experiment #1: Evaluation of Retinal Penetration of NACA, as amended July 2017 (Changed Initiation of treatment from P14 to P21 and changed termination point from P21 changed to P35 to match the time points in companion studies). The research was performed in 3 discrete experiments.

Evaluation of Retinal Penetration of NACA. To determine retinal levels of NACA/NAC after administration of 20 mg/mL NACA in drinking water for 7 days. This experiment will provide rapid confirmation that NACA and/or NAC penetrate the retina following oral administration. This experiment will also evaluate if the breakdown in the blood-retinal barrier in RP affects the levels of NACA in the retina by comparing animals with RP to wild type mice.

Rd10 and C57BL/6 wild type mice will begin treatment at P28 and at P35 mice will be euthanized and plasma, vitreous and retina samples will be sent to AITB for determination of NAC and NACA levels. Experimental groups: (n=10/group). The research was performed in 3 discrete experiments:
1. Rd10 NACA 20 mg/mL in drinking water measurements at P35;
2. Rd10 with no addition to drinking water measurements at P35;
3. C57/BL6 NACA 20 mg/mL in drinking water measurements at P35;
4. C57/BL6 with no addition to drinking water measurements at P35.

All levels of NACA and NAC were below the limit of quantification of the assay (BLQ) for mice which did not receive NACA in the drinking water. Nearly all measurements of NACA and NAC in aqueous, vitreous and retina were also BLQ and are not discussed.

The mean NACA levels in plasma for animals treated with 20 mg/ml in drinking water were greater than the NAC levels in both strains of mice (N=10 for each strain). The mean NACA levels and NAC levels were greater in rd10 mice than in the wild type. (FIG. 1).

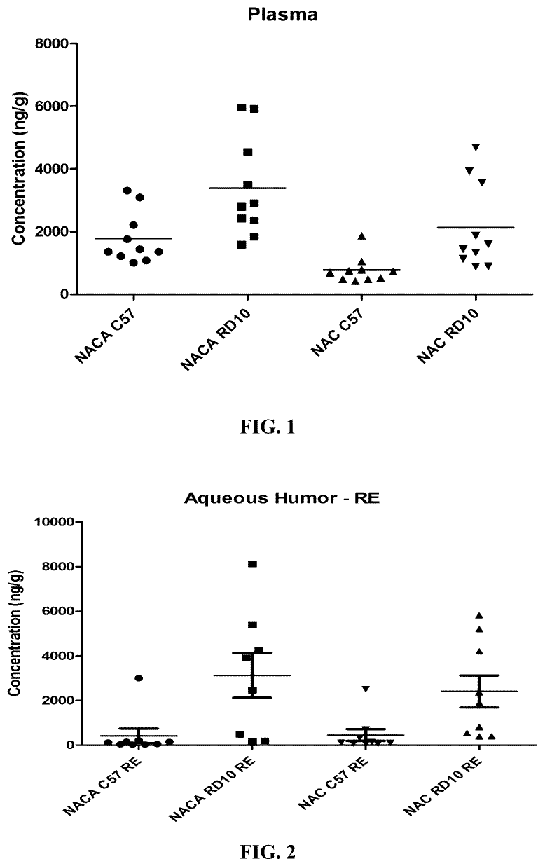

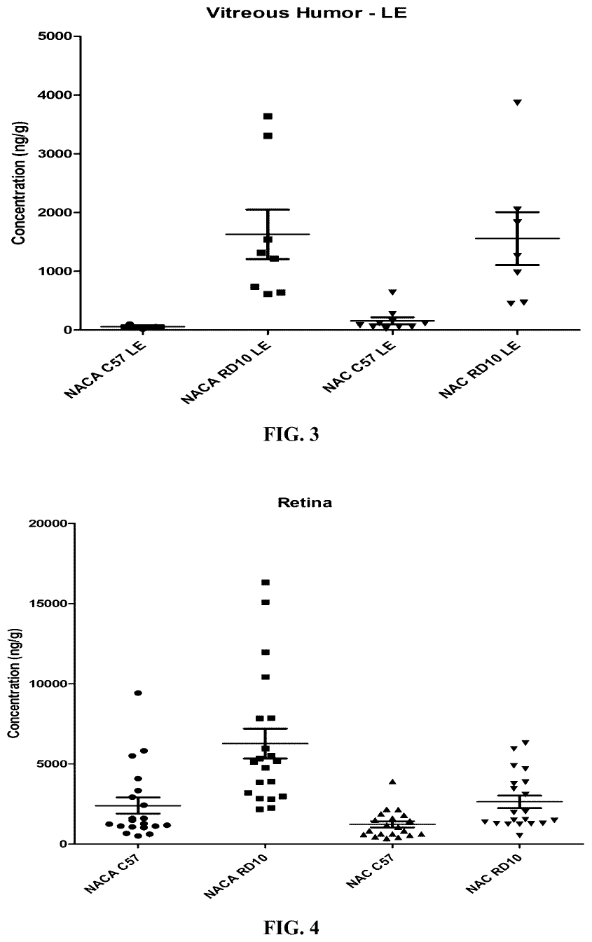

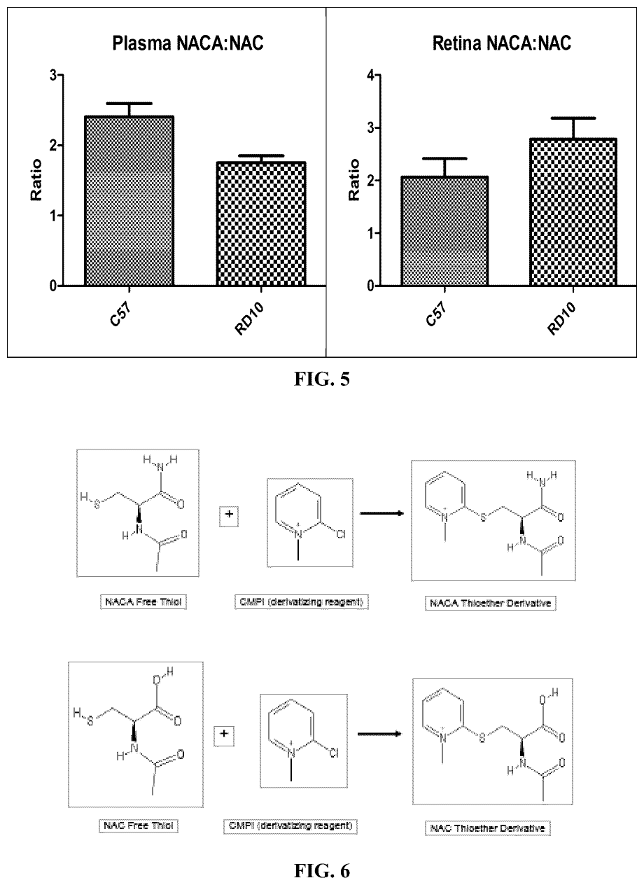

The mean NACA and NAC levels in aqueous humor were lower than in plasma. The mean concentration of NACA in the aqueous humor (N=8 eyes for rd10, N=10 for C57/B16) was also higher in rd10 mice than wild type. (FIG. 2). A similar pattern was observed for the vitreous humor (N=7 or 8 eyes for rd10, N=10 eyes for C57/B16). (FIG. 3). In the retina, NACA and NAC were measurable in all animals treated with NACA (N=20 eyes for each strain). The mean levels of NACA and NAC were higher than those observed in plasma (FIG. 4). The ratio of NACA: NAC in the retina indicates that NACA penetrated retina to a greater extent in rd10 mice than in C57/B16 mice (FIG. 5). FIG. 6 shows the thioether derivatives of NAC and NACA for LCMS analyses.

The data from this experiment demonstrate that NACA levels are measurable in the target tissue (the retina) following oral administration of 20 mg/mL in drinking water. It is estimated that mice consume 3-4 mL of water/day, leading to an estimated dose of NACA of 60-80 mg/day. These doses resulted in mean retina levels of approximately 6.5 μg/g in rd10 mice and 2.4 μg/g in wild type mice. It is notable that the ratio of NACA:NAC is greater in the retina of rd10 mice than in C57/B16 mice, suggesting that the disruption of the blood-retinal barrier in RP allows better penetration of NACA.

### Example 1: Nacuity Pharmaceuticals, Inc., Clinical Trial C-18-04, ‘SLO RP’

Oral NPI-001 tablets (250 mg, BID) significantly delayed progression of RP measured by photoreceptor cell preservation in participants with USH over 24 months compared with placebo tablets (FIG. 7). NPI-001 is the first drug to demonstrate significant preservation of photoreceptor cells in USH, representing an important gene-agnostic approach to address an unmet need in this sight-threatening disease (Kern and Wall, 2026).

FIG. 8 shows pharmacokinetics of NACA (NPI-001) orally dosed over 14 days in subjects 18 years and older.

### REFERENCES

Bharti K, den Hollander A I, Lakkaraju A, Sinha D, Williams D S, Finnemann S C, Bowes-Rickman C, Malek G, D'Amore P A. Cell culture models to study retinal pigment epithelium-related pathogenesis in age-related macular degeneration. Exp Eye Res. 2022 September; 222:109170. doi: 10.1016/j.exer.2022.109170. Epub 2022 Jul. 11. PMID: 35835183; PMCID: PMC9444976.
Datta S, Cano M, Ebrahimi K, Wang L, Handa J T. The impact of oxidative stress and inflammation on RPE degeneration in non-neovascular AMD. Prog Retin Eye Res. 2017 September; 60:201-218. doi: 10.1016/j.preteyeres.2017.03.002. Epub 2017 Mar. 20. PMID: 28336424; PMCID: PMC5600827.
Kern J R; Wall G M. NPI-001 Slows Photoreceptor Cell Loss in Adults with Retinitis Pigmentosa Associated with Usher Syndrome. Abstract accepted, Association for Research in Vision and Ophthalmology Meeting, Denver, C O, USA, May 3-7, 2026.
Raghu G, Berk M, Campochiaro P A, Jaeschke H, Marenzi G, Richeldi L, Wen F Q, Nicoletti F, Calverley PMA. The Multifaceted Therapeutic Role of N-Acetylcysteine (NAC) in Disorders Characterized by Oxidative Stress. Curr Neuropharmacol. 2021; 19 (8): 1202-1224. doi: 10.2174/1570159X19666201230144109. PMID: 33380301; PMCID: PMC8719286.

What is claimed is:

1. A method for treatment of age-related macular degeneration, geographic atrophy, glaucoma or diabetic retinopathy in an animal or human in need thereof, the method comprising:
providing an effective amount of an N-acetylcysteine amide (NACA) sufficient to inhibit degradation of vision.

2. The method of claim 1, wherein the eye disease is age-related macular degeneration.

3. The method of claim 1, wherein the eye disease is geographic atrophy.

4. The method of claim 1, wherein the eye disease is glaucoma.

5. The method of claim 1, wherein the eye disease is diabetic retinopathy.

6. The method of claim 1, wherein the NACA is provided orally, peritoneally, intravenously, dermally, bucally, sublingually, topically, topical ocularly, intraocularly, intravitreally, transmucosally, or by inhalation.

7. The method of claim 1, wherein the NACA inhibits the reduction in vision based on a primary endpoint, a secondary endpoint, or both.

8. The method of claim 1, wherein the NACA is at least one of:
dosed as multiple tablets per day;
dosed for at least 3 months, 6 months, or 12 months;
dosed for 24 months;
dosed for more than 24 months;
dosed in one or more tablets, each containing 200, 225, 250, 275, 300, 350, 400, 450, 500, 600, 700, 800, 900, or 1,000 mg NACA, once daily;
dosed in one or more tablets, each containing 200, 225, 250, 275, 300, 350, 400, 450, 500, 600, 700, 800, 900, or 1,000 mg NACA, twice daily;
dosed in one or more tablets, each containing 200, 225, 250, 275, 300, 350, 400, 450, 500, 600, 700, 800, 900, or 1,000 mg NACA, thrice daily;
dosed in one or more intravitreal implants, each containing 100, 200, 225, 250, 275, 300, 350, 400, 450, 500, 600, 700, 800, 900, or 1,000 μg diNACA, once every 3 months;
dosed in one or more intravitreal implants, each containing 100, 200, 225, 250, 275, 300, 350, 400, 450, 500, 600, 700, 800, 900, or 1,000 μg diNACA, once every 6 months; or
dosed in one or more intravitreal implants, each containing 100, 200, 225, 250, 275, 300, 350, 400, 450, 500, 600, 700, 800, 900, or 1,000 μg diNACA, once every year.

9. A method for treatment of age-related macular degeneration, geographic atrophy, glaucoma or diabetic retinopathy in an animal or human in need thereof, the method comprising:
providing an effective amount of (2R,2R′)-3,3′-disulfanediyl bis(2-acetamidopropanamide) (diNACA) sufficient to inhibit degradation of vision.

10. The method of claim 9, wherein the eye disease is age-related macular degeneration.

11. The method of claim 9, wherein the eye disease is geographic atrophy.

12. The method of claim 9, wherein the eye disease is glaucoma.

13. The method of claim 9, wherein the eye disease is diabetic retinopathy.

14. The method of claim 9, wherein the diNACA is provided orally, peritoneally, intravenously, dermally, bucally, sublingually, topically, topical ocularly, intraocularly, intravitreally, transmucosally, or by inhalation.

15. The method of claim 9, wherein the diNACA inhibits the reduction in vision based on a primary endpoint.

16. The method of claim 9, wherein the diNACA inhibits the reduction in vision based on a secondary endpoint.

17. The method of claim 9, wherein the diNACA is at least one of:
dosed as multiple tablets per day;
dosed for at least 3 months, 6 months, or 12 months;
dosed for 24 months;
dosed for more than 24 months;
dosed in one or more tablets, each containing 200, 225, 250, 275, 300, 350, 400, 450, 500, 600, 700, 800, 900, or 1,000 mg NACA, once daily;
dosed in one or more tablets, each containing 200, 225, 250, 275, 300, 350, 400, 450, 500, 600, 700, 800, 900, or 1,000 mg NACA, twice daily;
dosed in one or more tablets, each containing 200, 225, 250, 275, 300, 350, 400, 450, 500, 600, 700, 800, 900, or 1,000 mg NACA, thrice daily;
dosed for at least 6 months, or 12 months;
dosed for at least 24 months;
dosed in one or more intravitreal implants, each containing 100, 200, 225, 250, 275, 300, 350, 400, 450, 500, 600, 700, 800, 900, or 1,000 μg diNACA, once monthly; or
dosed in one or more intravitreal implants, each containing 100, 200, 225, 250, 275, 300, 350, 400, 450, 500, 600, 700, 800, 900, or 1,000 μg diNACA, once every 2 months.

18. The method of claim 9, wherein diNACA is dosed in one or more intravitreal implants, each containing 100, 200, 225, 250, 275, 300, 350, 400, 450, 500, 600, 700, 800, 900, or 1,000 μg diNACA, once every 3 months.

19. The method of claim 9, wherein diNACA is dosed in one or more intravitreal implants, each containing 100, 200, 225, 250, 275, 300, 350, 400, 450, 500, 600, 700, 800, 900, or 1,000 μg diNACA, once every 6 months.

20. The method of claim 9, wherein diNACA is dosed in one or more intravitreal implants, each containing 100, 200, 225, 250, 275, 300, 350, 400, 450, 500, 600, 700, 800, 900, or 1,000 μg diNACA, once every year.
